(19) United States
(12) Patent Application Publication
(10) Pub. No.: US 2005/0228626 A1
(43) Pub. Date: Oct. 13, 2005

(54) System and method for health analysis

(76) Inventor: Kim Simelius, Tampere (FI)

Correspondence Address: MORGAN & FINNEGAN, L.L.P., 3 WORLD FINANCIAL CENTER, NEW YORK, NY, 10281-2101, US

(21) Appl. No.: 10/817,198

(22) Filed: Apr. 2, 2004

(51) Int. Cl. G06G 7/48; G06G 7/58; A61K 31/42; A01N 43/80

(52) U.S. Cl. 703/11

(57) ABSTRACT

One or more personalized models of one or more of a user's organs, systems, and/or the like may, for example, be run, and/or biological measurement data may, for instance, be obtained for the user. Comparison and/or the like of model output and obtained biological data may, for instance, be performed. A condition of the user may, for example, be deduced.

### FIELD OF INVENTION

This invention relates to systems and methods for health analysis.

### BACKGROUND INFORMATION

In recent years, there has been an increase in the use of computers in various healthcare-related fields. For example, bioinformatics is increasingly being employed, computers are increasingly being employed where physical files, papers, films, and/or the like were once employed, and computers are increasingly being employed in diagnosis, procedures, and the like.

Accordingly, there may be interest in technologies that, for example, provide for the use of computers in healthcare-related fields.

### SUMMARY OF THE INVENTION

In various embodiments, one or more personalized models of one or more of a user's organs, systems, and/or the like may be run, and/or biological measurement data may be obtained for the user. Comparison and/or the like of model output and obtained biological data may, in various embodiments, be performed.

It is further noted that, in various embodiments, a condition of the user may be deduced.

### BRIEF DESCRIPTION OF THE DRAWINGS

FIG. 1 shows an exemplary personalized thorax model.

FIG. 2 shows an exemplary personalized heart model.

FIG. 3 shows an exemplary personalized cardiac conduction system model.

FIG. 4 shows information regarding exemplary operational modes.

FIG. 5 is a diagram showing exemplary steps involved in model use.

FIG. 6 shows an exemplary electrocardiogram sensor placement.

FIG. 7 shows an exemplary waveform comparison technique.

FIG. 8 shows an exemplary simulated activation sequence.

FIG. 9 shows exemplary simulated body surface potential maps.

FIG. 10 shows exemplary simulated electrocardiogram and vectorcardiogram output.

FIG. 11 shows an exemplary computer.

FIG. 12 shows a further exemplary computer.

### DETAILED DESCRIPTION OF THE INVENTION

General Operation

According to various embodiments one or more personalized models of one or more of a user's organs, systems, and/or the like may run, for example, on the user's wireless node and/or other computer, one or more servers and/or the like (e.g., remote servers and/or the like), and/or the like. Such a model might, according to various embodiments, be capable of running in one or more operational modes, simulating one or more normal states, simulating one or more abnormal states, and/or the like.

Biological measurement data may, in various embodiments, be obtained for the user. According to various embodiments, output produced by one or more of the user's personalized models is compared to the biological measurement data.

It is further noted that, in various embodiments, one or more of the user's personalized models may be placed into one or more selected operational modes such that, for example, output produced by one or more of the models matches biological measurement data obtained for the user. In various embodiments, a condition of the user may be deduced in view of, for instance, such selected operational modes.

Various aspects of the present invention will now be discussed in greater detail.

Personalized Model Provision

As alluded to above, according to various embodiments of the present invention one or more personalized models of one or more of a user's organs, systems, and/or the like may be provided for a user. Such functionality may be implemented in a number of ways. For instance, the user might act to visit a doctor's office, hospital, physiology laboratory, and/or the like to have one or more such personalized models placed on her wireless node and/or other computer, one or more servers and/or the like, and/or the like.

Such personalized models could be created in a number of ways. For example, medical imaging data (e.g., magnetic resonance imaging (MRI) data, ultrasound data, x-ray data, and/or the like), 3D modeling, measurement data (e.g., electrocardiogram (ECG) data, electroencephalogram (EEG) data, magnetocardiogram data, and/or the like), expert knowledge, and/or the like might be employed. It is noted that, in various embodiments, a personalized model could model the specificities of an actual organ, system, and/or the like of a user.

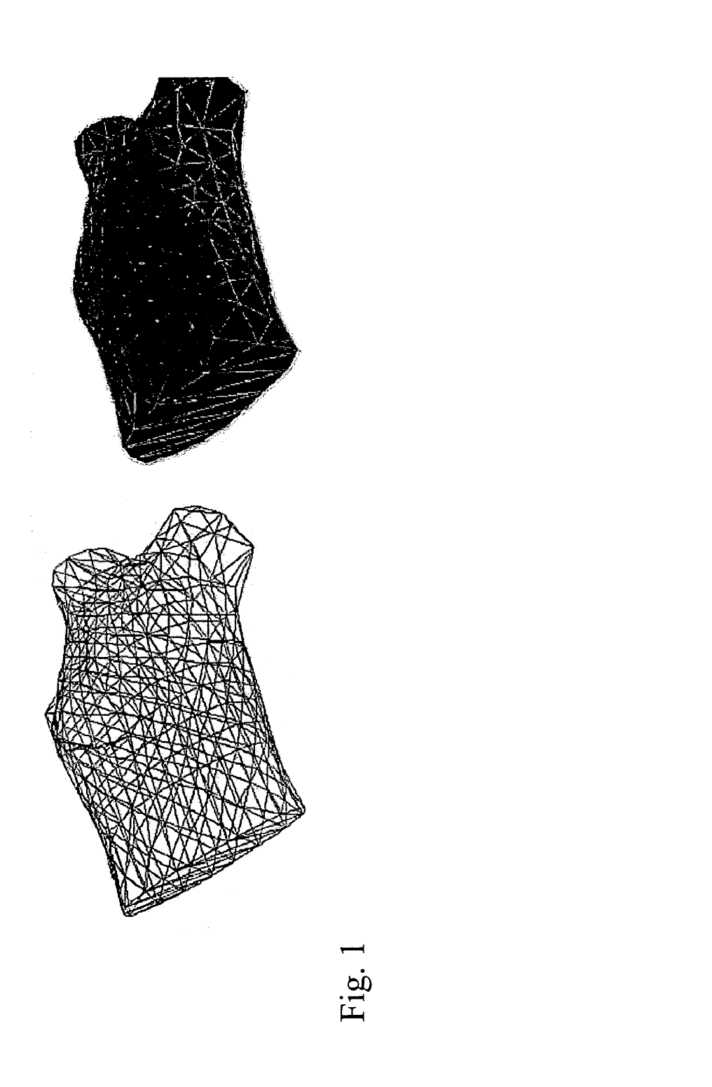

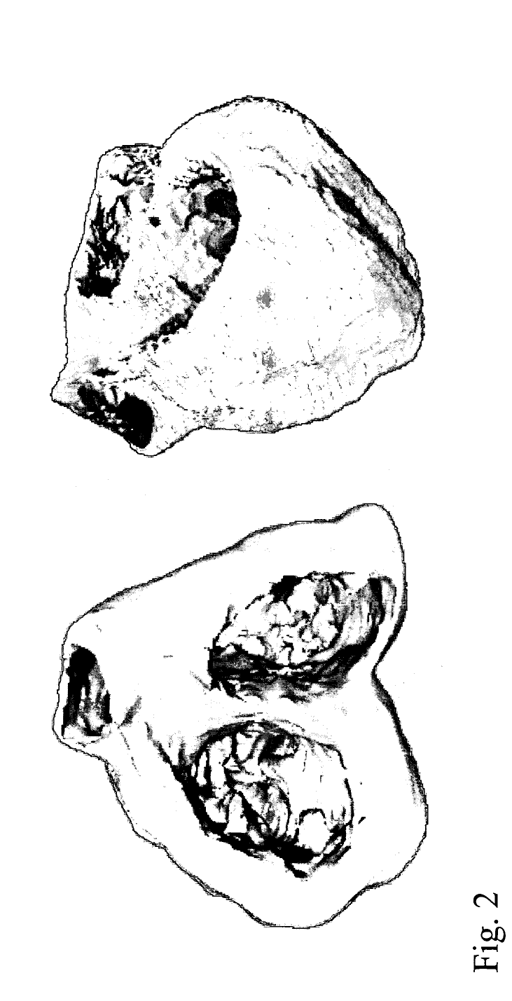

Various personalized models might be created. For example, a personalized thorax model, a personalized heart model, and/or a personalized cardiac conduction system model might be created. Exemplary such models employable in various embodiments of the present invention are shown in FIGS. 1-3.

As further examples, a personalized brain model, a personalized pancreas model, a personalized kidney model, a personalized lung model, and/or other personalized organ model could alternately or additionally be created. As additional examples, a personalized nervous system model, a personalized endocrine system model, a personalized renal system model, a personalized cardiopulmonary system model, and/or the like could alternately or additionally be created.

It is noted that, according to various embodiments of the present invention, such a personalized model could be calibrated. For instance, calibration of a personalized model could be performed such that output produced by the model is made to be like that of corresponding biological measurement data to be obtained from the user (e.g., via action of the user's node and/or a peripheral device thereof).

In, for example, the case where a personalized model is to create ECG-type output and a reduced lead set ECG reading (e.g., a two lead or three lead reading) is to be obtained from the user, calibration of the personalized model could, in various embodiments, be performed such that output of the model would be like that of a reduced lead set ECG reading (e.g., a two lead or three lead reading).

It is noted that, in various embodiments, calibration may be carried out at a doctor's office, hospital, physiology laboratory, and/or the like. Such a facility might, in various embodiments, have equipment for more detailed recordings of the user's signals than the equipment that the user carries with her. For example, in various embodiments in the calibration phase it may be possible to record 128 channels of ECG from the user and then tune a heart model to reproduce all those 128 ECG signals whereby, for instance, calibration could be done perhaps more reliably than by using a few leads only. The number of ECG sensors carried by the user could, in various embodiments, be a subset of the larger number of ECG channels (e.g., 128).

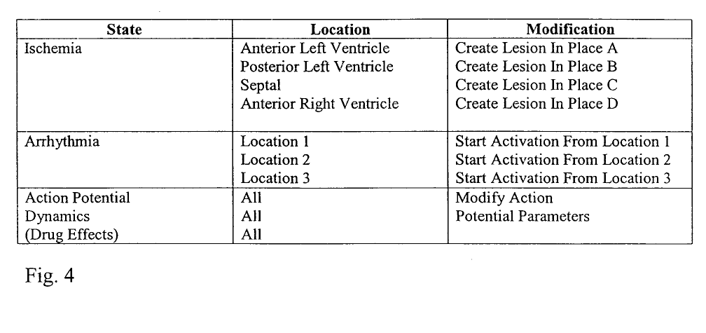

As indicated above, in various embodiments a personalized model could be capable of running in one or more operational modes, simulating one or more normal states, simulating one or more abnormal states, and/or the like. Such functionality could be implemented in a number of ways. For example, as shown in FIG. 4, an operational mode corresponding to an ischemia abnormal state could be implemented, for instance, by including in the model the ability to simulate one or more lesions at one or more locations.

As another example, as shown in FIG. 4, an operational mode corresponding to an arrhythmia abnormal state could be implemented, for instance, by including in the model the ability to simulate one or more activations starting at one or more locations. As yet another example, as shown in FIG. 4, an operational mode corresponding to an action potential dynamic (e.g., a drug effect) abnormal state could be implemented, for instance, by including in the model the ability to simulate appropriate modification of one or more action potential parameters.

Further abnormal states for which operation modes could be implemented could, in various embodiments, include conduction disorders, endocrine disorders (e.g., diabetic disorders), neurological disorders, and/or the like.

Additional modeling information is provided later herein.

Operation

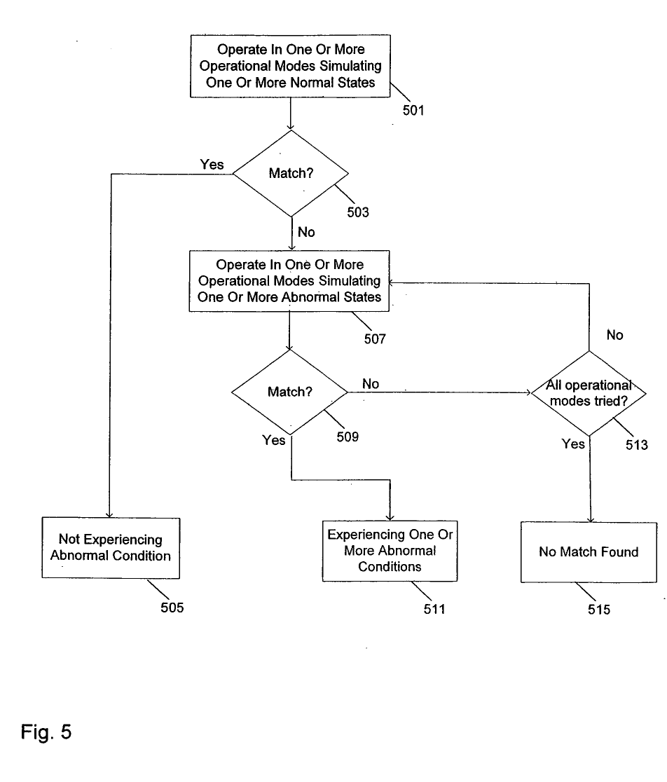

With respect to FIG. 5 it is noted that, according to various embodiments of the present invention, one or more personalized models of the sort discussed above may be operated in one or more operational modes simulating one or more normal states (step 501) while biological measurement data is obtained for the user.

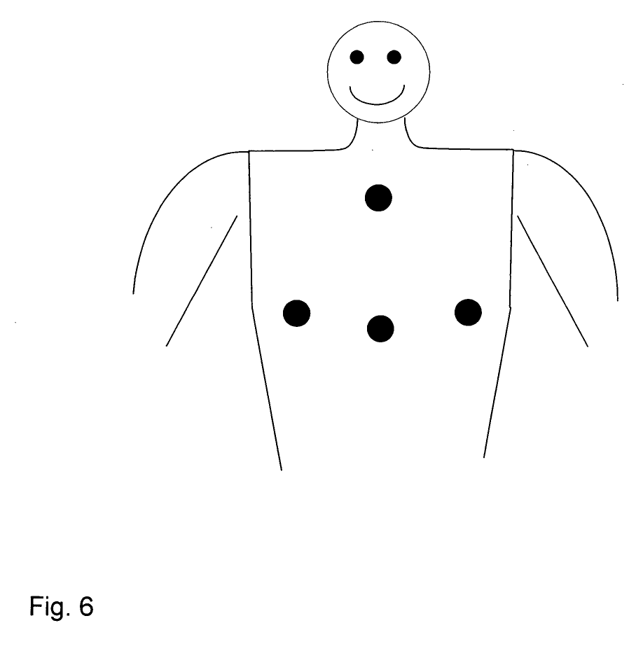

Such biological measurement data could be obtained (e.g., by the user's node and/or other computer, one or more servers and/or the like, and/or the like) in a number of ways. For example, one or more ECG sensors, one or more EEG sensors, one or more molecular sensors (e.g., hormonal sensors), one or more ionic concentration sensors, one or more neurological sensors, and/or the like could be employed. Shown in FIG. 6 is an exemplary ECG sensor placement according to various embodiments of the present invention.

It is noted that, in various embodiments, one or more of the sensors could be implanted in the user. It is further noted that, in various embodiments, the sensors might, for example, communicate with the user's node and/or other computer, one or more servers and/or the like, and/or the like, such communication perhaps being via one or more peripheral devices, and/or the like.

Communication between the user's node and/or other computer, one or more servers and/or the like, and/or the like and such sensors and/or peripherals might, for instance, employ Bluetooth, WiFi (e.g., IEEE 802.11b and/or IEEE 802.11g), Ultra Wide Band (UWB), Universal Serial Bus (USB), IEEE 1394, IEEE 1394b, and/or the like. Such UWB might, for instance, employ IEEE 802.15a, IEEE 802.15.3, and/or the like. In various embodiments, commercially-available sensors and/or peripherals may be utilized.

With further respect to FIG. 5 it is noted that, in various embodiments, a determination could be made as to whether or not output produced by one or more of the personalized models matched biological measurement data obtained for the user (step 503). Such matching could be performed in a number of ways.

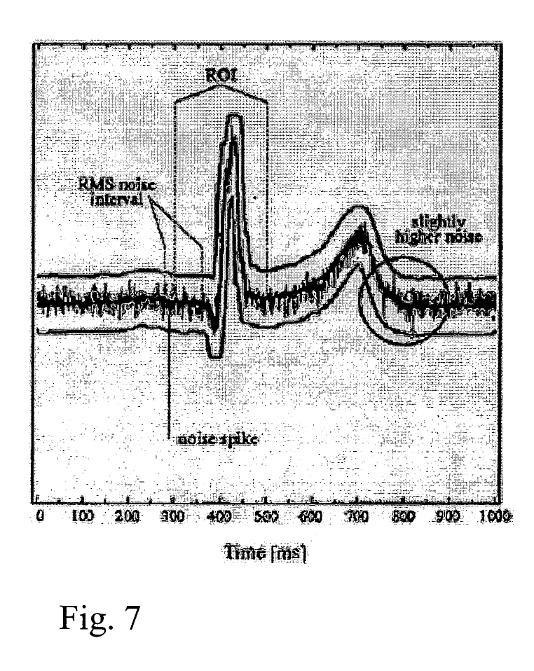

For instance, one or more signal analysis techniques, waveform comparison techniques, and/or the like could be employed. Such techniques could, for example, involve the superposition and/or differencing of output produced by one or more of the personalized models and biological measurement data obtained for the user. In various embodiments, a tube technique, such as one in accordance with FIG. 7, could be employed. The signal analysis could, in various embodiments, be carried out with the help of neural networks and/or the like. For instance, backpropagation neural networks, learning vector quantization classifiers, and/or self-organizing maps might be employed.

It is noted that, in various embodiments, one or more parameters could specify, for example, one or more thresholds and/or the like corresponding to what would be considered a match. Such parameters might, for instance, be set by an expert (e.g., a physician and/or physiologist), a system administrator, and/or the like.

Determination could, in various embodiments, be made as to the extent to which output produced by one or more of the personalized models deviated from biological measurement data obtained for the user. Such functionality could, in various embodiments, be implemented via one or more signal analysis techniques, waveform comparison techniques, and/or the like.

In the case where match was found, the user could, in various embodiments, be considered to not be experiencing an abnormal condition (step 505). In the case where match was not found, one or more personalized models could, in various embodiments, be placed in one or more operational modes simulating one or more abnormal states (step 507). In various embodiments, a determination could be made as to whether or not output produced by one or more of the personalized-models now matched biological measurement data obtained for the user (step 509).

It is noted that, in various embodiments, one or more available operational modes simulating one or more abnormal states could be iteratively applied until a match was found and/or until all available operational modes simulating abnormal states had been employed without a match being found.

In the case where match was found, the user could, in various embodiments, be considered to be experiencing one or more abnormal conditions corresponding to one or more of the operational states (step 511). In various embodiments, in the case where all available operational modes simulating abnormal states had been tried without a match being found, recordation, indication, and/or the like could be made that no-match had been found (steps 513, 515).

As alluded to above, one or more of the various operations depicted in FIG. 5 might, for instance, be performed on a user's wireless node and/or other computer, one or more servers and/or the like, and/or the like.

According to various embodiments of the present invention, one or more operations could be performed with respect to steps 505, 511, and/or 515. For instance, corresponding indication could be displayed to the user, be provided to one or more remote locations, be provided to one or more remote servers and/or the like, be provided to a personal health monitoring system used in physical training and/or the like, be provided to a patient monitoring center, system, and/or the like, and/or the like.

Such indication could be provided in a number of ways. For example, a graphical user interface (GUI) and/or other interface (e.g., one provided by the user's node and/or other computer), Simple Object Access Protocol (SOAP), Remote Method Invocation (RMI), Java Messaging Service (JMS), one or more telephone calls employing voice synthesis, and/or the like could be employed.

Indication could, in various embodiments, include specification of one or more abnormal conditions believed to be affecting the user, such specification in various embodiments including details regarding such abnormal conditions. Among such details might, for example, be indication of one or more predicted lesion locations in the case where the user is believed to be experiencing ischemia, indication of one or more activation start locations in the case where the user is believed to be experiencing arrhythmia, indication of one or more action potential parameter modifications and/or specification of one or more suspected drugs in the case where the user is believed to be experiencing an action potential change, and/or the like.

It is noted that, in various embodiments, preprocessing may be performed on biological measurement data obtained for the user. It is further noted that, in various embodiments, recording, analysis, simulation, and/or the like is arranged to be available on a device that the user carries with her and that, in various embodiments, there is no need for the user to go to a doctor's office, hospital, physiology laboratory, and/or the like for, for instance, health analysis.

In various embodiments, there is, for example, no large system for collecting measurement data. Moreover, in various embodiments there is, for example, no measurement data collected to a central location.

It is noted that, in various embodiments, recording, analysis, simulation, and/or the like is carried out totally and/or partially on the user's wireless node and/or other computer. It is further noted that, in various embodiments, recording, analysis, simulation, and/or the like could totally and/or partially be carried out on one or more servers and/or the like. Accordingly, for example, computational load on the user's wireless node and/or other computer could be reduced. Such might be desirable, for instance, in the case where computational power of the user's wireless node and/or other computer is limited.

In various embodiments, distributed recording, analysis, simulation, and/or the like could be implemented wherein, for example, some or all recording, analysis, simulation, and/or the like is carried out on one or more servers and/or the like (e.g., powerful back-end computers). For instance, in various embodiments more computationally-intensive parts might be run on one or more servers and/or the like (e.g., powerful back-end computers), whereas less computationally-intensive parts and/or control might be run on the user's wireless node and/or other computer.

Additional Cardiac Modeling Information

Further to that which is discussed above, various models, modeling techniques, aspects, and/or the like applicable, perhaps with modifications thereto, in various embodiments of the present invention will now be discussed.

In various embodiments a ventricular model of the human heart that produces correct normal activation can be used, for example, to simulate the effects of ischemia and infarction or arrhythmias involving the conduction network. Such a model may, for instance, feature a realistic anatomical structure, including intramural fiber rotation, and a physiologically sensible model of the conduction process.

Turning to models of cardiac activation, it is noted that cardiac activation has been modeled on several levels. Action potential models like the Beeler-Reuter model and the Luo-Rudy model aim at describing the ionic currents as accurately as possible. According to various embodiments, a cardiac activation model may have anisotropy due to fibrous structure, realistic fiber geometry and intramural fiber rotation, correct boundary conditions, and/or the like. It is further noted that, in various embodiments, to reproduce correct activation of the normal heart and correct electrocardiographic signals, the model may feature realistic anatomy and fiber structure, have a proper conduction system, and/or be anatomically correctly positioned within a thorax model. Such models can, in various embodiments, also be used to simulate activation in abnormal conditions.

It is noted that, in various embodiments, it may be viewed as important to correctly select cell and/or tissue models depending on the application of the model, and/or model geometry may be considered important. In various embodiments, a bidomain model may be employed, such a model perhaps being modified so that it is able to produce some effects that the traditional bidomain model cannot, for example by adding discrete elements to mimic the behavior of gap junctions or by incorporating directional differences to action potential shape.

In various embodiments the effects of choosing the diffusion coefficient D—the conductivities of various models of the myocardium—may be considered. The value of D may, in various embodiments, be considered to relate to phenomena like ischemia, cell-to-cell decoupling, decay into fibrillation and re-entrant arrhythmias. In various embodiments, monodomain models may be considered accurate enough to determine activation patterns.

Turning to action potential models, it is noted that, as is known in the art, a Beeler-Reuter action potential model incorporates four ionic currents: the inward sodium current i_{Na}, a slow inward current iₛ mostly carried by calcium ions, a time-dependent outward potassium current iₖ, and a time dependent outward current iₓₗ mostly carried by potassium ions. As is also known in the art, the Luo-Rudy model is a model of the ionic currents of the guinea pig heart. The model features more-than fifteen ionic currents, also incorporating the currents of the sarcoplasmic reticulum.

As is known in the art, the effect of ischemic conditions on the cell-to-cell conduction has been simulated using the Luo-Rudy model. The simulations were carried out for a fiber of 70 serially arranged Luo-Rudy cells connected by gap junctions. It was found that by applying realistic potassium concentration elevation, hypoxia, and acidosis, a conduction block could be induced.

Turning to models of cardiac tissue and numerics, it is noted that, as known in the art, the effect of the boundary conditions on intracellular current has been considered by comparing two alternative formulations for the boundary condition. The first condition proposed for the intracellular current is that it vanishes at the boundary to the extracardiac space, that is, the sealed-end condition. The second condition proposed for the intracellular current is that it is equal to the membrane current at the boundary. It was found that the two boundary conditions give essentially equal results when the space constant is large compared to the cell size.

As is also known in the art, the use of an eikonal model in slab simulations has been demonstrated, with it being demonstrated that the depth of the initial stimulus can be deduced from the shape of the epicardial potentials. The importance of incorporating sources due to the anisotropy of the cardiac muscle was stressed.

As is also known in the art, it has been found factors in cardiac activation include the anisotropy due to the shape of the cardiac cells, rotation of the fiber direction from the epicardium to the endocardium, the obliqueness of the fibers compared to the epicardial surface, and the effect of the conduction system.

Study of different conductivity values reported for the bidomain conductivities has, as known in the art, pointed out an inconsistency in the measurement, suggesting that a modeler might, perhaps, be able to virtually pick the conductivities to be used at will.

As is known in the art, although traditional monodomain models may not predict differences in action potential shape attributed to the direction of propagation compared to the fiber direction, such behavior has been observed in real cardiac tissue. The observed differences in the maximum rate of change of the membrane potential during upstroke and the shape of the action potential foot were attributed to the membrane capacitance being dependent on direction of propagation. As is also known in the art, it has been hypothesized that the cell-to-cell connections at the intercalated disks explain the differences in apparent directional capacitance and load observed by the cell.

A numerically stable method for solving partial differential equations in a highly irregular and evolving grid has, as is known in the art, been presented. Problems that are solved by this method may come up, for instance, if movement is taken into account in the heart model and the activating elements are in relative movement. In various embodiments, a Braun natural-neighbor influence function, and/or a modification thereof, may be employed in calculation of nodal excitation where, for example, all neighbors of the cell are allowed to influence the potential change in the center cell.

As is known in the art, a method for simulating cardiac conduction with a model that has an irregular grid has been described, with changes in the potential of a cell being computed by taking into account the contribution of each 6 facets of the element, and using 18 points around each facet to determine the current flow through the facet.

As is also known in the art, the response of a two-dimensional excitable tissue slab to a stimulus slightly off the surface has been computed, suggesting that a heart model can be used to predict responses to externally applied electrical current. Such may be applicable, for example, in modeling, defibrillator shocks.

Turning to heart models, it is noted that known in the art is the Miller-Geselowitz model, wherein action potential is modeled as a simple activation step followed by linear repolarization segments. Despite the simplicity of the model, the electrocardiograms produced by the model for the normal activation are in line with measured data (e.g., normal body surface potential maps and electrocardiograms). As is also known in the art, ischemic regions have been created in the Miller-Geselowitz model by modifying the action potentials and assigning an activation delay to the elements in the ischemic region, producing results consistent with recorded data from patients with ischemic heart disease. The computation of body surface potentials and magnetic fields from the simulated cardiac sources has, as is known in the art, been described, with it being argued as to why the anisotropy of the cardiac muscle is not significant in computing the body surface potentials. It is noted, however, that in various embodiments modeling may be performed with the view that anisotropy may not be essential, but that neglecting it provides clear differences from normal electrocardiograms and magnetocardiograms.

The Pollard-Barr model is, as is known in the art, perhaps one of the first realistic models for the human conduction system. The model is built using data recorded from the human heart to come up with the proper activation times and a geometrical mapping of data on the anatomy of the conduction system on the heart model, and output of the model shows marked similarity to measured data in the activation pattern.

The Lorange model, as is known in the art, is constructed from anatomical data of a human heart, wherein the fiber structure is generated by nesting ellipsoids in the ventricular walls and assigning realistically rotating fiber orientation. The model also features a simplistic conduction system that is able to reproduce initial activation sites. The body surface potentials are computed by embedding the individual dipole sources into an inhomogeneous torso model. One application of this model is the use of so-called thorax extension method, whereby the anisotropic skeletal muscle layer below the surface of the torso is replaced by a thicker isotropic tissue. As us known in the art, Lorange-type models have been successfully employed in simulating normal electrocardiograms, electrocardiograms resulting from a conduction block and ectopic beats, with the results having been validated against clinical data. The Lorange model, and/or a modification thereof, may be employed in various embodiments of the present invention, such employment perhaps improving the robustness of the surface potentials to small variations in activation.

The Dubé heart model has, as known in the art, been employed in simulating ischemia. As is known in the art; the model can produce normal electrocardiograms and electrocardiograms from a heart with an occlusion in any of the major arteries, with results being in good agreement with literature data and measured data.

The Berenfeld model, as is known in the art, is based on the FitzHugh-Nagumo action potential model in a heart model with cubical lattice and a cell spacing of 1 mm. As is known in the art, with regard to the Berenfeld model, the effect of the FitzHugh-Nagumo model parameters to the action potential shape and considerations on the effects of rotational anisotropy have been presented. In the core of the model, regular three-by-three differentiation formulas are used, wherein the second cross derivatives are computed by using the four corner cells in the plane of the differentiation.

The Winslow model, as is known in the art, combines a cell model and anatomically accurate geometric model where fiber geometry has been obtained from diffusion tensor magnetic resonance imaging (DTMRI). Mäkelä models, as is known in the art, have been implemented using modern anatomical imaging methods like deformable models for cardiac source imaging. Apart from source imaging, anatomical models obtained in this manner can, for example, be applied to model the cardiac activation individually. By, for example, using deformable models and accurate fiber geometry, both an individualized heart model and an individualized model of the thorax may, in various embodiments, be obtained.

The Sermesant model, as is known in the art, involves mechanical contraction being coupled to electrical activation. The model uses a FitzHugh-Nagumo action potential model and a geometric model with 256 nodes. The exact activation pattern may remain somewhat unclear, although good results in the mechanical contraction as compared to imaging data from the beating heart have been reported.

The He-Li model, as is known in the art, can be employed, for example, in localizing the origin of cardiac activity, for carrying out activation time mapping, and for determining the transmembrane potential distribution in the heart. The He-Li model, in one aspect, imposes goodness-of-fit measures to the propagated activation and the simulated body surface potentials and to apply optimization to achieve the a match.

It is noted that, in various embodiments, anisotropic properties may be considered important in modeling extracardiac fields. For example, in various embodiments an intramural source may not generate any electric signal without an anisotropic component.

As is known in the art, directional differences have been observed in bidomain models that have equal anisotropy ratios for the intracellular and extracellular spaces. As is also known in the art, the importance of the axial current component in the formation of body surface potential maps has been demonstrated; in earlier uniform double layer models this contribution did not exist, as the double layer was uniform. In various embodiments of the present invention, the contribution of the axial current component may be viewed as a feature for producing realistic body surface potential maps from simulated normal activation.

In various embodiments, a propagation model including 2,000,000 excitable elements comprising the conduction system and the myocardium, and 8,000,000 non-excitable elements making up the intra- and extracardiac volumes may be employed. The elements may, for example, be located on a cubic lattice with 0.5-mm spacing. The geometry may, for example be reconstructed from photographic images (e.g., images of 1-mm frozen slices of the human heart). The assignment of the principal fiber direction may, for instance, be performed separately for left-ventricular, right-ventricular and papillary-muscle cells, with the fiber direction rotating from endocardium to epicardium.

A hybrid model of the ventricular myocardium describing the subthreshold behavior of the elements according to the anisotropic bidomain theory, while in the suprathreshold region having the elements behave as cellular automata, is one example of a model that may be employed. Such a model may, for example, include 2×10⁶ excitable elements on, for instance, a cubic lattice with 0.5-mm spacing. Each element may, for example be assigned a specific type and a vector of local fiber direction. During simulation, the elements may, for example, undergo a series of state transitions. Their electrotonic interactions may, for example, be governed by the generalized cable equation which, for example, is derived under an assumption of equal anisotropy ratios. Values known in the art may, for example, be employed for model physiological parameters.

Extracardiac fields may, for example, be computed such that the anisotropy in the cardiac muscle is taken into account. The body surface potential maps may, for example, be computed separately for the isotropic and the axial component of the source dipoles to, for example, evaluate the effect of the anisotropy on the body surface potentials, perhaps with subsequent summing of the two potential maps with a weighting. A realistic body surface potential map (BSPM) sequence may, for example, be produced by employing a weighting of 2:1 of the isotropic and axial potential. Electrocardiograms and vectorcardiograms may, for example, be computed using the nodes closest to corresponding ECG electrodes for the limb leads and/or the construction of the Wilson Central Terminal (WCT).

Modeling may, for example, involve employment of fiber geometry that results in simulated body surface potential maps, computed from propagated activation that results from artificial pacing stimulus in various locations of the ventricles, agreeing well with clinically recorded data. The employed fiber structure may, for instance, be macroscopically realistic, and/or the propagated excitation may tend to reproduce true activation of the human heart in the case of catheter pacing. Furthermore, modeling may, for example, be such that calculations of body surface potentials produce realistic results.

It is further noted that modeling may, for example, involve employment of fiber geometry such that the accessory pathways in Wolff-Parkinson-White patients can be localized, and/or so that the effect of fiber rotation through the wall of cardiac muscle on epicardial potentials can be demonstrated.

Turning to conduction system models it is noted that the cardiac conduction system may, for example, be modeled to produce correct body surface electromagnetic fields (e.g., electrocardiograms and/or magnetocardiograms) and/or activation rather than, for instance, strictly following a predetermined anatomical pattern. A computer program for the interactive editing of the conduction system may, for example, be created and/or employed. Such a program might, for example, employ OpenGL and/or the like. It is further noted that such a program might, for example, request a user to select a surface on which the conduction system will be designed, and/or may allow for surface modification. A triangulated surface of the intracavitary blood masses may, for example, be created from corresponding volumes of a ventricular model.

Such software might, for example, allow a user to create nodes on the surface (e.g., by pointing to the desired location), and/or connect those nodes (e.g., define the connection matrix for the nodes). Moreover such software might, for example, allow for later repositioning of the nodes, and/or for modification of the connection matrix. It is further noted that the nodes could, for example, be automatically named to represent their location, perhaps with the names reflecting their functionality (e.g., some nodes are connection points for the conduction system, while some are Purkinje-myocardial junction sites (PMJs) where the activation enters the myocardium).

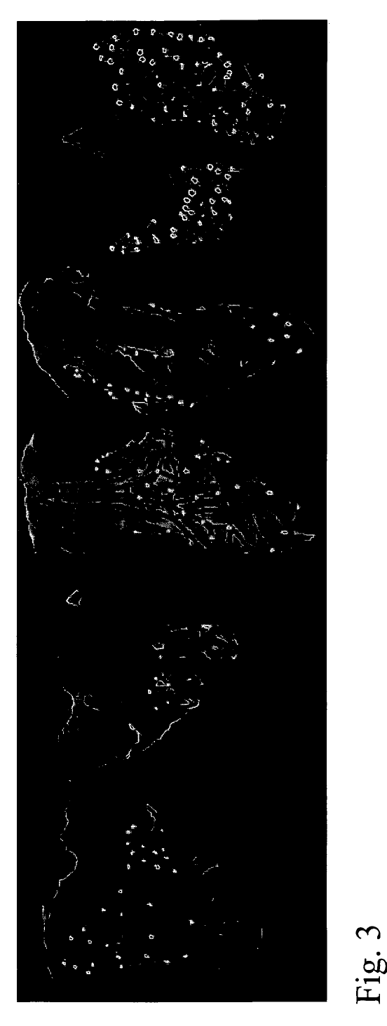

Turning again to FIG. 3, exemplary conduction system geometry will be discussed. As shown in FIG. 3, the His bundle consists of a single branch in the right ventricle, whereas it resembles a fan-like sheet of fibers in the left ventricle. The His bundle in FIG. 3 continues on both sides as the Purkinje network that contains the Purkinje-myocardial junction (PMJ) sites. With further respect to FIG. 3 it is noted that, on the right, a prominent feature of the conduction system is the single bundle that carries the activation from the septum to the free wall and the papillary muscles along the moderator band. With still further respect to FIG. 3 it is noted that, on the left, there are three major areas of activation: the septum, the inferior free wall, and the superior free wall. In the exemplary conduction system shown in FIG. 3, there is no conduction network in the posterior free wall.

The volume model for simulations may, for example, be created on a 1.5-mm thick endocardial layer by first projecting the nodes onto this layer and then tracing the connections. The thickness of the connections and PMJ sites may, for example, be adjusted to ensure connectivity and proper propagation between the conduction system and the myocardium. By, for example, changing the conductivity properties, the propagation velocity may, for instance, be adjusted to approximately 2.0 m/s. The activation time of a PMJ site may, for example, be defined by the propagation through the conduction system. The volume model may, for example, be superimposed on the model of the ventricular myocardium at the beginning of the simulation.

With regard to simulation of normal activation it is noted that a number of simulations may, for example, be run to iteratively determine correct parameters for the model employed (e.g., an anisotropic bidomain model), to create an anatomically correct conduction system, and/or to investigate the effects of geometry on the electromagnetic fields.

A reasonable set of parameters may, for example, first be chosen for the initial analyses on the anatomy of the conduction system. Then, as the conduction system produced a good match with experimental invasive data, the parameters could, for example, be fine-tuned to give correct QRS duration and timing for the breakthroughs. Initial modifications to the thorax geometry could, for example, then be made, perhaps by comparing the location and orientation of the heart in the thorax model to anatomical textbooks and imaging data. Perhaps after some adjustments to the conduction system, the geometry could, for example, be finalized using the vectorcardiogram as a guide, while perhaps still keeping the geometry within anatomically normal limits.

The simulations could, for example, be run with a time step of 50 μs (time steps between 5 μs and 100 μs may, for example, all yield consistent isochrones, and the value of 50 μs may, for example, be chosen as a compromise between numerical accuracy and computation speed). Correct QRS duration may, for example, be achieved by using surface-to-volume ratio χ=1200 cm⁻¹, axial conductivity σ₁=2.5 mS/cm, and transverse conductivity σ₁=0.5 mS/cm.

The initial shape of the conduction system may, for example, be built to match various anatomical descriptions. Modifications may, for example, later be made, for instance, to balance the timing of different directions of initial activation. Such might be achieved, for example, by adjusting the basal location of the His bundle, by changing the length of the right bundle branch by altering its course, and/or by modification of the left bundle branches. The thickness of the fibers and/or the spatial extent of the Purkinje myocardial junctions may, for example, be varied. Such modifications may, for example, provide for achievement of a reasonable match with measured activation sequences of the human heart.

In the case where the geometry of the ventricles is taken from an abnormal subject (e.g., a victim of a traffic accident) rather than from the geometry of the thorax (e.g., a normal volunteer), the heart may, for example, need to be refitted inside a thorax model manually. Such could be achieved in a number of ways. For instance, a cardiologist might, perhaps with the aid of a 3D manipulator program, determine a normal position and orientation of the heart within the thorax. Alternately or additionally, a normal position might be determined in light of anatomical textbooks, expert descriptions, cardiac imaging data, and/or the like.

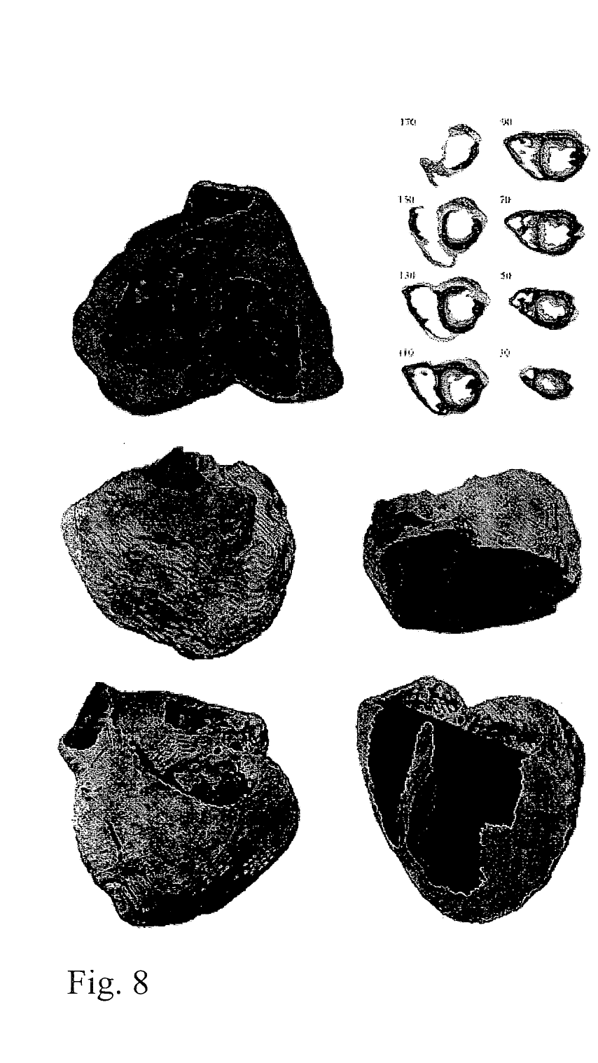

The exemplary simulated activation sequence shown in FIG. 8 agrees with isochrones obtained from an isolated human heart. The ventricular activation starts in the left ventricular septum (layer 110), matched by a right ventricular septal activation 20 ms later (layers 70-90). Almost simultaneously with the RV septal activation, the inferior (in body coordinates) and anterior LV activation appear (layers 90-110). These are followed by the activation of the RV free wall (layers 90-130). The RV and LV breakthroughs take place at 30 ms and 45 ms, respectively. In the final stages of the QRS, activation propagates through the posterior LV free wall and the pulmonary conus.

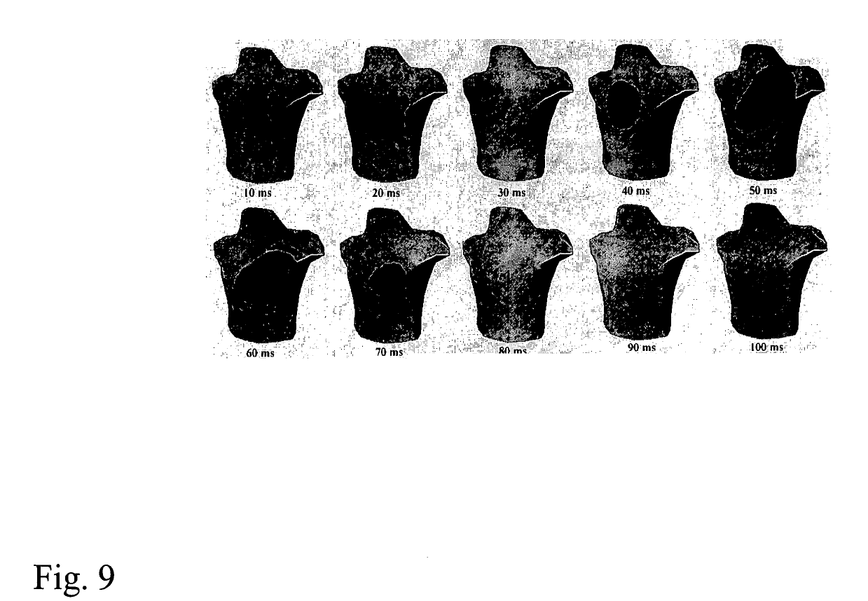

FIG. 9 shows exemplary simulated BSPMs. The initial maximum resulting from left septal activation is anterior and slightly superior. Then, the minimum on the back moves upward and travels over the right shoulder onto the right anterosuperior region, indicating apical activation and masking of the left septal activation by the corresponding right septal activation. The area of positive potentials then drifts to the back, as the right ventricular breakthrough happens, and the activation in the left ventricle travels mainly to the posterior direction. Finally, a positive area appears in the high posterior area, resulting from the activation of the pulmonary conus.

Magnetocardiographic (MCG) maps on a planar surface above the chest may, for, example, be computed. The sensors in MCG recording may, for example, be arranged similarly (e.g., to facilitate comparison). Due to the geometry, the MCG may, for example, “see” primarily the sources that are parallel to the frontal plane. The MCG may, for example, be sensitive to activation wavefronts that are close to the sternum and moving in the frontal plane, whereas other currents (e.g., deeper and other directions) may need to be much stronger to be picked up by the MCG to the same extent.

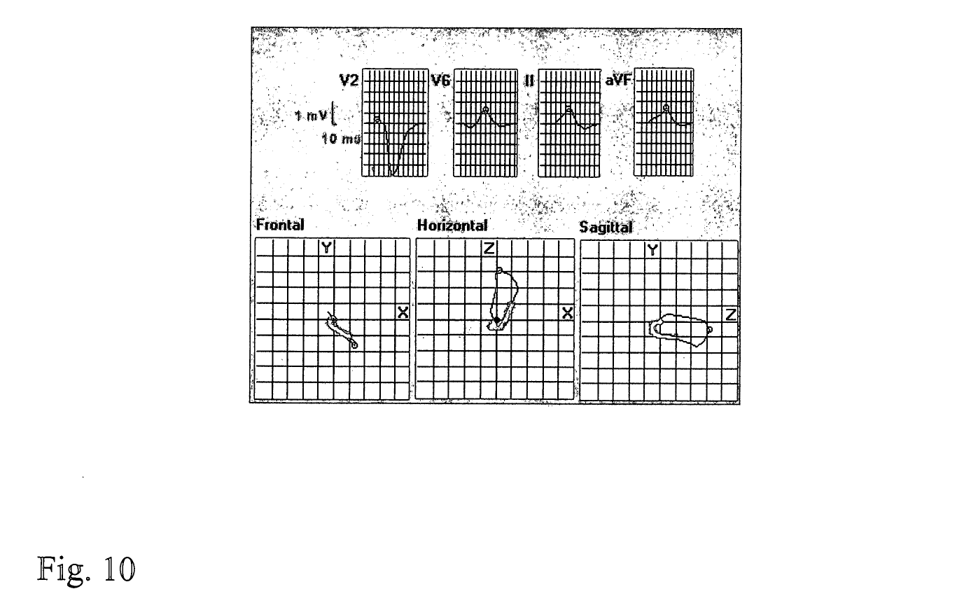

With respect to FIG. 10 it is noted that at least the prominent features of a 12-lead electrocardiogram and/or vectorcardiogram may, for example, be produced by model output. The signal morphology in the chest leads of the 12-lead electrocardiogram correctly shows an increase in the R wave amplitude from lead V1 to V6, and simultaneously a decrease in the S wave amplitude. The crossover from prominent S to prominent R morphology takes place between V4 and V5. The augmented limb leads show the following features: the aVR is mainly negative, and the aVF mainly positive, while the aVL has a slightly negative but very unclear morphology. The aVR displays abnormal late positivity. This is reflected to the leads I, II and III that are otherwise normal. The vectorcardiogram shows a tight clockwise loop pointing to low right in the frontal plane, the frontal angle being approximately 60°. In the horizontal plane, the wide counterclockwise loop points mainly in the left posterior direction. The sagittal loop is counterclockwise and points to low posterior direction.

It is noted that information from anatomical and electrophysiological studies on the conduction system are to some extent contradictory. For example, bundle branch blocks are usually functional phenomena, where the anatomy of the conduction may be completely intact. Moreover, very different conduction system anatomies often produce similar normal ECGs. Accordingly, it may, for example, be desirable to differentiate between true anatomical defects and functional anomalies (e.g., the electrophysiologically meaningful concept of left hemiblocks often has no basis in anatomy, especially in the ischemic heart).

It may, for example, be desirable to determine whether simplifying the modeling of anisotropic properties by the use of equal anisotropy ratios affects the propagation significantly. Moreover, it might, for example, be desirable to, in model implementation, assume that anisotropy has little effect on activation, although it may be important in the forward computation. However, more fundamental features like transmurally varying cell properties may, for example, mask the effects of anisotropy, especially in the repolarization phase. Therefore, it may, for example, be desirable to consider the effect of action potential heterogeneity (M-cells).

It is noted that the model-produced activation sequence, compared to the activation sequence measured from an isolated human heart, may, for example, show the simulated activation of the left ventricle to very well match a recorded one. Moreover, the simulated body surface potential pattern may, for example, be found to at least mostly correspond to recorded BSPMs. In the case where deviance is found with the initial positive area on the anterior chest moving slightly too quickly to the back, and/or the late activation (positive) of the pulmonary conus being too strong on the anterior chest, such deviance may, for example, be found to be attributable to inaccurate positioning of the ventricular model within the thorax. Where model output has the MCG transition corresponding to right ventricular breakthrough taking place in a different manner than in recorded data, such may, for example, be found to be due to neglecting the anisotropic properties of the thorax.

The general outline on a simulated 12-lead ECG may, for example, be normal but, corresponding to the deviation in the BSPM, perhaps appear rotated to the left, and/or the crossover in chest lead morphology may take place between V4 and V5 instead of between V3 and V4 as usual. The strong pulmonary activation may, for example, be reflected as an abnormal late positivity in the aVR lead. Model vectorcardiogram output may, for example, be completely in the normal limits, the frontal angle being, for example, close to 60°. It is noted that the horizontal loop may, for example, be oriented slightly too posteriorly.

It is noted that, even with model algorithm simplifications, simulation output may, for example, produce correct activation sequences, electrocardiogram, and/or magnetocardiograms.

The anatomy of the conduction system might, for example, be not restricted. Such might be the case, for example, due to lack of anatomical information of the Purkinje network. Still, the model may, for example, be implemented so as to be consistent with the anatomic literature. It is further noted that the modeled conduction system may, for example, found to be robust, at least insofar as small changes in the shape of the conduction system producing only unnoticeable or small effects on produced electrocardiograms and/or the like.

Hardware and Software

Various operations and/or the like described herein may be executed by and/or with the help of computers. Further, for example, devices described herein may be and/or may incorporate computers. The phrases “computer”, “general purpose computer”, and the like, as used herein, refer but are not limited to a processor card smart card, a media device, a personal computer, an engineering workstation, a PC, a Macintosh, a PDA, a portable computer, a computerized watch, a wired or wireless terminal, phone, node, and/or the like, a server, a network access point, a network multicast point, a set-top box, a personal video recorder (PVR, a game console, or the like, perhaps running an operating system such as OS X, Linux, Darwin, Windows CE, Windows XP, Windows Server 2003, Palm OS, Symbian OS, or the like, perhaps employing the Series 60 Platform and/or Series 90 Platform, and perhaps having support for Java and/or .Net.

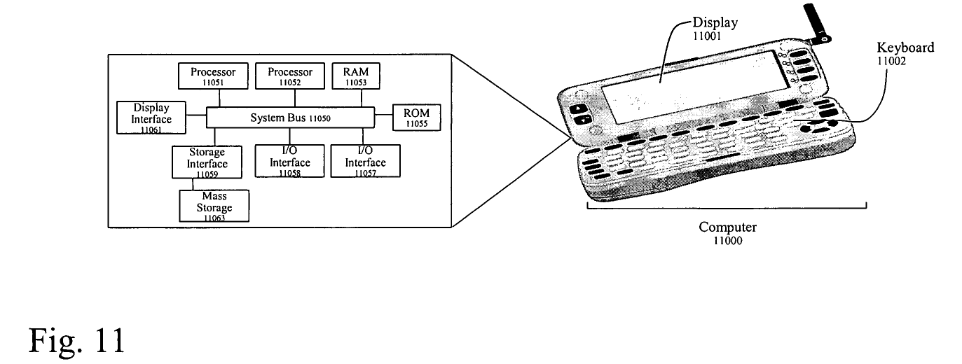

The phrases “general purpose computer”, “computer”, and the like also refer, but are not limited to, one or more processors operatively connected to one or more memory or storage units, wherein the memory or storage may contain data, algorithms, and/or program code, and the processor or processors may execute the program code and/or manipulate the program code, data, and/or algorithms. Accordingly, exemplary computer 11000 as shown in FIG. 11 includes system bus 11050 which operatively connects two processors 11051 and 11052, random access memory 11053, read-only memory 11055, input output (I/O) interfaces 11057 and 11058, storage interface 11059, and display interface 11061. Storage interface 11059 in turn connects to mass storage 11063. Each of I/O interfaces 11057 and 11058 may be an Ethernet, IEEE 1394, IEEE 1394b, IEEE 802.11a, IEEE 802.11b, IEEE 802.11g, IEEE 802.11i, IEEE 802.11e, IEEE 802.11n, IEEE 802.15a, IEEE 802.16a, IEEE 802.16d, IEEE 802.16e, IEEE 802.16x, IEEE 802.20, IEEE 802.15.3, ZigBee, Bluetooth, terrestrial digital video broadcast (DVB-T), satellite digital video broadcast (DVB-S), digital audio broadcast (DAB), general packet radio service (GPRS), Universal Mobile Telecommunications Service (UMTS), DVB-H, IrDA (Infrared Data Association), and/or other interface known in the art.

Mass storage 11063 may be a hard drive, optical drive, or the like. Processors 11051 and 11052 may each be a commonly known processor such as an IBM or Motorola PowerPC, an AMD Athlon, an AMD Opteron, an Intel ARM, an Intel XScale, a Transmeta Crusoe, a Transmeta Efficeon, an Intel Xenon, an Intel Itanium, or an Intel Pentium. Computer 11000 as shown in this example also includes a touch screen 11001 and a keyboard 11002. In various embodiments, a mouse, keypad, and/or interface might alternately or additionally be employed. Computer 11000 may additionally include or be attached to card readers, DVD drives, floppy disk drives, and/or the like whereby media containing program code (e.g., for performing various operations and/or the like described herein) may be inserted for the purpose of loading the code onto the computer.

In accordance with various embodiments of the present invention, a computer may run one or more software modules designed to perform one or more of the above-described operations. Such modules might, for example, be programmed using languages such as Java, Objective C, C, C#, C++, Perl, and/or Xen according to methods known in the art. Corresponding program code might be placed on media such as, for example, DVD, CD-ROM, and/or floppy disk. It is noted that any described division of operations among particular software modules is for purposes of illustration, and that alternate divisions of operation may be employed. Accordingly, any operations discussed as being performed by one software module might instead be performed by a plurality of software modules. Similarly, any operations discussed as being performed by a plurality of modules might instead be performed by a single module. It is noted that operations disclosed as being performed by a particular computer might instead be performed by a plurality of computers. It is further noted that, in various embodiments, peer-to-peer and/or grid computing techniques may be employed.

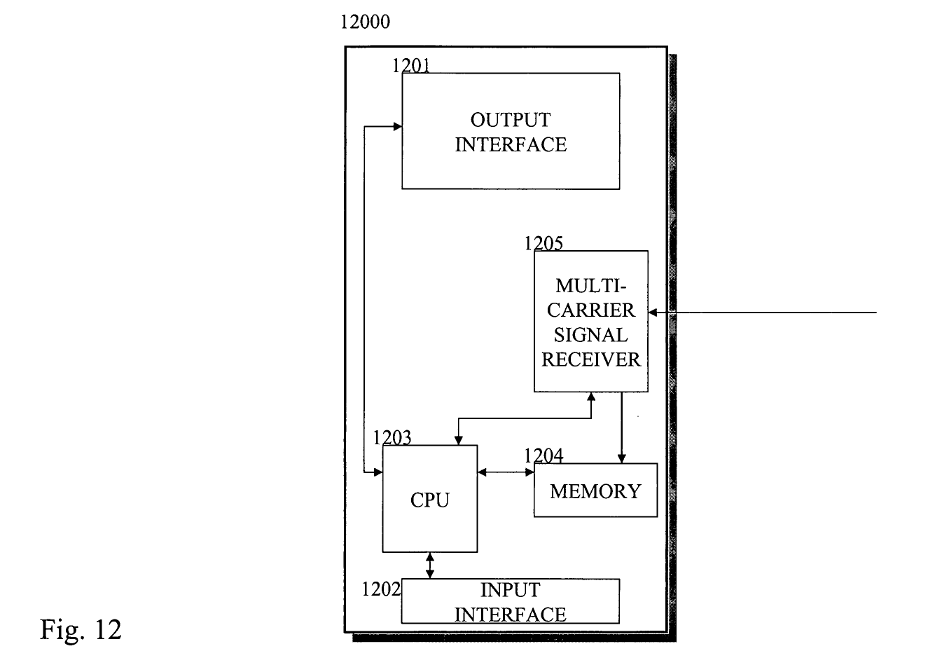

Shown in FIG. 12 is a block diagram of a terminal, an exemplary computer employable in various embodiments of the present invention. The terminal of FIG. 12 has been discussed in the foregoing. In the following, corresponding reference signs have been applied to corresponding parts. Terminal 12000 of FIG. 12 may be used in any/all of the embodiments described herein. The terminal 12000 comprises a processing unit CPU 1203, a multi-carrier signal terminal part 1205 and a user interface (1201, 1202). The multi-carrier signal terminal part 1205 and the user interface (1201, 1202) are coupled with the processing unit CPU 1203. One or more direct memory access (DMA) channels may exist between multi-carrier signal terminal part 1205 and memory 1204. The user interface (1201, 1202) comprises a display and a keyboard to enable a user to use the terminal 12000. In addition, the user interface (1201, 1202) comprises a microphone and a speaker for receiving and producing audio signals. The user interface (1201, 1202) may also comprise voice recognition (not shown).

The processing unit CPU 1203 comprises a microprocessor (not shown), memory 1204 and possibly software. The software can be stored in the memory 1204. The microprocessor controls, on the basis of the software, the operation of the terminal 12000, such as the receiving of the data stream, the tolerance of the impulse burst noise in the data reception, displaying output in the user interface and the reading of inputs received from the user interface. The operations are described above. The hardware contains circuitry for detecting the signal, circuitry for demodulation, circuitry for detecting the impulse, circuitry for blanking those samples of the symbol where significant amount of impulse noise is present, circuitry for calculating estimates, and circuitry for performing the corrections of the corrupted data.

Still referring to FIG. 12, alternatively, middleware or software implementation can be applied. The terminal 12000 can be a hand-held device which the user can comfortably carry. Advantageously, the terminal 12000 can be a cellular mobile phone which comprises the multi-carrier signal terminal part 1205 for receiving the multicast transmission stream. Therefore, the terminal 12000 may possibly interact with the service providers.

Ramifications and Scope

Although the description above contains many specifics, these are merely provided to illustrate the invention and should not be construed as limitations of the invention's scope. Thus it will be apparent to those skilled in the art that various modifications and variations can be made in the system and processes of the present invention without departing from the spirit or scope of the invention.

1. A method comprising:
operating a simulated organ;
obtaining biological measurement data for a user;
selecting an operational mode for the simulated organ such that an output signal produced by the simulated organ matches the biological measurement data; and
deducing a condition of the user, wherein deducing takes into account the selected operational mode.

2. The method of claim 1, wherein the simulated organ is personalized to model specificities of an actual organ of the user.

3. The method of claim 1, wherein the simulated organ is calibrated such that the output signal produced by the simulated organ shares one or more characteristics with the biological measurement data.

4. The method of claim 3, wherein the simulated organ is calibrated such that the output signal produced by the simulated organ is like that of a reduced lead set electrocardiogram reading.

5. The method of claim 1, wherein the simulated organ is simulated at a wireless node.

6. The method of claim 1, wherein the simulated organ is simulated at a server.

7. The method of claim 1, wherein a plurality of organs are simulated.

8. The method of claim 7, wherein a thorax is simulated.

9. The method of claim 1, wherein the condition corresponds to an abnormal state.

10. The method of claim 9, wherein the abnormal state relates to an organ of the user.

11. The method of claim 10, wherein the simulated organ simulates the organ of the user.

12. The method of claim 9, wherein the abnormal state is arrhythmia.

13. The method of claim 9, wherein the abnormal state is a conduction disorder.

14. The method of claim 9, wherein the abnormal state an endocrine disorder.

15. The method of claim 1, wherein the deduced condition corresponds to a normal state.

16. The method of claim 15, wherein the normal state corresponds to an organ of the user.

17. The method of claim 16, wherein the simulated organ simulates the organ of the user.

18. The method of claim 1, wherein preprocessing is performed on the biological measurement data.

19. The method of claim 1, wherein the simulated organ simulates a heart.

20. The method of claim 1, wherein the simulated organ simulates a pancreas.

21. The method of claim 1, wherein the simulated organ simulates a brain.

22. The method of claim 1, wherein one or more sensors are employed in obtaining the biological measurement data.

23. The method of claim 22, wherein one or more of the sensors are electrocardiogram sensors.

24. The method of claim 23, wherein six electrocardiogram sensors are employed.

25. The method of claim 23, wherein three electrocardiogram sensors are employed.

26. The method of claim 22, wherein one or more of the sensors are electroencephalogram sensors.

27. The method of claim 22, wherein one or more of the sensors are molecular sensors.

28. The method of claim 22, wherein one or more of the sensors are ionic concentration sensors.

29. The method of claim 1, wherein waveform comparison is employed.

30. The method of claim 1, wherein the user is informed of the deduced condition.

31. The method of claim 1, wherein one or more servers are informed of the deduced condition.

32. The method of claim 1, wherein a plurality of operational modes for the simulated organ are iteratively selected.

33. The method of claim 1, wherein a body system is simulated.

34. The method of claim 33 wherein an endocrine system is simulated.

35. The method of claim 33 wherein a renal system is simulated.

36. The method of claim 33 wherein a cardiopulmonary system is simulated.

37. A method comprising:
operating a simulated organ;
obtaining biological measurement data for a user; and
comparing an output signal produced by the simulated organ with the biological measurement data,
wherein the simulated organ is personalized to model specificities of an actual organ of the user.

38. The method of claim 37, further comprising determining the output signal produced by the simulated organ to deviate from the biological measurement data.

39. The method of claim 37, wherein the simulated organ is calibrated such that the output signal produced by the simulated organ shares one or more characteristics with the biological measurement data.

40. The method of claim 39, wherein the simulated organ is calibrated such that the output signal produced by the simulated organ is like that of a reduced lead set electrocardiogram reading.

41. The method of claim 37, wherein the simulated organ is simulated at a wireless node.

42. The method of claim 37, wherein the simulated organ is simulated at a server.

43. The method of claim 37, wherein a plurality of organs are simulated.

44. The method of claim 43, wherein a thorax is simulated.

45. The method of claim 37, wherein preprocessing is performed on the biological measurement data.

46. The method of claim 37, wherein the simulated organ simulates a heart.

47. The method of claim 37, wherein the simulated organ simulates a pancreas.

48. The method of claim 37, wherein the simulated organ simulates a brain.

49. The method of claim 37, wherein one or more sensors are employed in obtaining the biological measurement data.

50. The method of claim 49, wherein one or more of the sensors are electrocardiogram sensors.

51. The method of claim 50, wherein six electrocardiogram sensors are employed.

52. The method of claim 50, wherein three electrocardiogram sensors are employed.

53. The method of claim 49, wherein one or more of the sensors are electroencephalogram sensors.

54. The method of claim 49, wherein one or more of the sensors are molecular sensors.

55. The method of claim 49, wherein one or more of the sensors are ionic concentration sensors.

56. The method of claim 37, wherein waveform comparison is employed.

57. The method of claim 37, wherein a plurality of operational modes for the simulated organ are iteratively selected.

58. The method of claim 37, wherein a body system is simulated.

59. The method of claim 58 wherein an endocrine system is simulated.

60. The method of claim 58 wherein a renal system is simulated.

61. The method of claim 58 wherein a cardiopulmonary system is simulated.

62. The method of claim 37, further comprising deducing a condition of the user.

63. A system comprising:
a memory having program code stored therein; and
a processor disposed in communication with the memory for carrying out instructions in accordance with the stored program code;
wherein the program code, when executed by the processor, causes the processor to perform:
operating a simulated organ;
obtaining biological measurement data for a user;
selecting an operational mode for the simulated organ such that an output signal produced by the simulated organ matches the biological measurement data; and
deducing a condition of the user, wherein deducing takes into account the selected operational mode.

64. The system of claim 63, wherein the simulated organ is personalized to model specificities of an actual organ of the user.

65. The system of claim 63, wherein the simulated organ is calibrated such that the output signal produced by the simulated organ shares one or more characteristics with the biological measurement data.

66. The system of claim 65, wherein the simulated organ is calibrated such that the output signal produced by the simulated organ is like that of a reduced lead set electrocardiogram reading.

67. The system of claim 63, wherein the simulated organ is simulated at a wireless node.

68. The system of claim 63, wherein the simulated organ is simulated at a server.

69. The system of claim 63, wherein a plurality of organs are simulated.

70. The system of claim 69, wherein a thorax is simulated.

71. The system of claim 63, wherein the condition corresponds to an abnormal state.

72. The system of claim 71, wherein the abnormal state relates to an organ of the user.

73. The system of claim 72, wherein the simulated organ simulates the organ of the user.

74. The system of claim 71, wherein the abnormal state is arrhythmia.

75. The system of claim 71, wherein the abnormal state is a conduction disorder.

76. The system of claim 71, wherein the abnormal state an endocrine disorder.

77. The system of claim 63, wherein the deduced condition corresponds to a normal state.

78. The system of claim 77, wherein the normal state corresponds to an organ of the user.

79. The system of claim 78, wherein the simulated organ simulates the organ of the user.

80. The system of claim 63, wherein preprocessing is performed on the biological measurement data.

81. The system of claim 63, wherein the simulated organ simulates a heart.

82. The system of claim 63, wherein the simulated organ simulates a pancreas.

83. The system of claim 63, wherein the simulated organ simulates a brain.

84. The system of claim 63, wherein one or more sensors are employed in obtaining the biological measurement data.

85. The system of claim 84, wherein one or more of the sensors are electrocardiogram sensors.

86. The system of claim 85, wherein six electrocardiogram sensors are employed.

87. The system of claim 85, wherein three electrocardiogram sensors are employed.

88. The system of claim 84, wherein one or more of the sensors are electroencephalogram sensors.

89. The system of claim 84, wherein one or more of the sensors are molecular sensors.

90. The system of claim 84, wherein one or more of the sensors are ionic concentration sensors.

91. The system of claim 63, wherein waveform comparison is employed.

92. The system of claim 63, wherein the user is informed of the deduced condition.

93. The system of claim 63, wherein one or more servers are informed of the deduced condition.

94. The system of claim 63, wherein a plurality of operational modes for the simulated organ are iteratively selected.

95. The system of claim 63, wherein a body system is simulated.

96. The system of claim 95 wherein an endocrine system is simulated.

97. The system of claim 95 wherein a renal system is simulated.

98. The system of claim 95 wherein a cardiopulmonary system is simulated.

99. A system comprising:
a memory having program code stored therein; and
a processor disposed in communication with the memory for carrying out instructions in accordance with the stored program code;
wherein the program code, when executed by the processor, causes the processor to perform:
operating a simulated organ;
obtaining biological measurement data for a user; and
comparing an output signal produced by the simulated organ with the biological measurement data,
wherein the simulated organ is personalized to model specificities of an actual organ of the user.

100. The system of claim 99, wherein the processor further performs determining the output signal produced by the simulated organ to deviate from the biological measurement data.

101. The system of claim 99, wherein the simulated organ is calibrated such that the output signal produced by the simulated organ shares one or more characteristics with the biological measurement data.

102. The system of claim 101, wherein the simulated organ is calibrated such that the output signal produced by the simulated organ is like that of a reduced lead set electrocardiogram reading.

103. The system of claim 99, wherein the simulated organ is simulated at a wireless node.

104. The system of claim 99, wherein the simulated organ is simulated at a server.

105. The system of claim 99, wherein a plurality of organs are simulated.

106. The system of claim 105, wherein a thorax is simulated.

107. The system of claim 99, wherein preprocessing is performed on the biological measurement data.

108. The system of claim 99, wherein the simulated organ simulates a heart.

109. The system of claim 99, wherein the simulated organ simulates a pancreas.

110. The system of claim 99, wherein the simulated organ simulates a brain.

111. The system of claim 99, wherein one or more sensors are employed in obtaining the biological measurement data.

112. The system of claim 111, wherein one or more of the sensors are electrocardiogram sensors.

113. The system of claim 112, wherein six electrocardiogram sensors are employed.

114. The system of claim 112, wherein three electrocardiogram sensors are employed.

115. The system of claim 111, wherein one or more of the sensors are electroencephalogram sensors.

116. The system of claim 111, wherein one or more of the sensors are molecular sensors.

117. The system of claim 111, wherein one or more of the sensors are ionic concentration sensors.

118. The system of claim 99, wherein waveform comparison is employed.

119. The system of claim 99, wherein a plurality of operational modes for the simulated organ are iteratively selected.

120. The system of claim 99, wherein a body system is simulated.

121. The system of claim 120 wherein an endocrine system is simulated.

122. The system of claim 120 wherein a renal system is simulated.

123. The system of claim 120 wherein a cardiopulmonary system is simulated.

124. The system of claim 99, wherein the processor further performs deducing a condition of the user.

125. An article of manufacture comprising a computer readable medium containing program code that when executed causes a wireless terminal to perform:
operating a simulated organ;
obtaining biological measurement data for a user;
selecting an operational mode for the simulated organ such that an output signal produced by the simulated organ matches the biological measurement data; and
deducing a condition of the user, wherein deducing takes into account the selected operational mode.

126. An article of manufacture comprising a computer readable medium containing program code that when executed causes a wireless terminal to perform:
operating a simulated organ;
obtaining biological measurement data for a user; and
comparing an output signal produced by the simulated organ with the biological measurement data,
wherein the simulated organ is personalized to model specificities of an actual organ of the user.
